(19) United States
(12) Patent Application Publication
(10) Pub. No.: US 2006/0195193 A1
(43) Pub. Date: Aug. 31, 2006

(54) Implant for closing an opening in the annulus fibrosus

(75) Inventors: Wilhelm Bloemer, Unteruhldingen-Muehlhofen (DE); Robert Schultz, Tuttlingen (DE); Michael Mayer, Graefelfing (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/360,986

(22) Filed: Feb. 22, 2006

Related U.S. Application Data
(63) Continuation of application No. PCT/EP04/08567, filed on Jul. 30, 2004.

(30) Foreign Application Priority Data
Aug. 26, 2003 (DE) 103 40 150.4

(51) Int. Cl. A61F 2/44

(52) U.S. Cl. 623/17.16

(57) ABSTRACT

In an implant in the form of a plug for closing an opening in the annulus fibrosus, in order to ensure as reliable a fixing of the plug as possible and a secure closure, it is proposed that the plug have rigid anchoring elements for the end plates of adjacent vertebrae on its upper face and on its lower face, and a deformable structure in the area between upper face and lower face.

This application is a continuation of international application number PCT/EP2004/008567 filed on Jul. 30, 2004.

The present disclosure relates to the subject matter disclosed in international application number PCT/EP2004/008567 of Jul. 30, 2004 and German application number 103 40 150.4 of Aug. 26, 2003, which are incorporated herein by reference in their entirety and for all purposes.

### BACKGROUND OF THE INVENTION

The invention relates to an implant in the form of a plug for closing an opening in the annulus fibrosus.

The spinal column in humans, in particular, the intervertebral discs, is subject to a natural aging process, in which the fluid content of the gel-like core of the intervertebral disc (nucleus pulposus) diminishes. As a result, the intervertebral disc undergoes a loss in height, and an increasing amount of strain is placed on the fibrous ring surrounding the nucleus (annulus fibrosus). Cracks may form in the annulus, through which nucleus material is pressed outwards. This initially causes protrusions (prolapse), with the nucleus material still remaining within the intervertebral disc. If the nucleus material has leaked completely from the annulus, this is referred to as a sequestrum. The amount of pain caused by prolapse and sequestrum may differ according to the extent to which the adjacent nerve roots or the spinal canal are affected.

During intervertebral disc surgery, all of the leaked nucleus material is, therefore, first removed. To ensure that another prolapse of the intervertebral disc will not occur, part of the nucleus still located within the intervertebral disc is also removed (nucleotomy). The thus removed material may be used for restoration or regeneration of the nucleus by part of the nucleus material removed from the patient during the nucleotomy being used to biotechnologically grow cells. An intervertebral disc cell transplant is produced from cells from the patient's own body and is injected into the patient's intervertebral disc again about two weeks after the intervertebral disc surgery. The injected cells settle again and contribute towards regeneration of the still existing nucleus.

However, a problem arises from the annulus no longer being intact. The injected cells may escape again through the opening made during the nucleotomy and through cracks in the annulus and die off before ingrowth was able to take place.

It is known to close openings in the annulus by inserting annulus closures (WO 01/10316 A1; WO 99/04720; WO 02/080821; WO 01/28464). In the majority of cases, the closure device used is directly supported on the annulus tissue and is sutured, adhesively bonded or joined in some other way thereto. Since the annulus tissue is a very flexible tissue, the closure is also subjected to the movements of the annulus. This closure is therefore at risk of becoming detached unintentionally. In exceptional cases, the closure device is also secured to the adjacent vertebrae. However, these are then exclusively net-like or membrane-like closures which are intended to close the opening in the annulus in the fashion of a foil. There is no guarantee of reliable sealing here. The object of the invention is to so design a generic implant that, on the one hand, a secure fixing of the implant and, on the other hand, a reliable closure of the opening in the annulus are ensured, while the movability of the annulus is to be maintained.

### SUMMARY OF THE INVENTION

This object is accomplished, in accordance with the invention, in an implant of the kind described at the outset by the plug having rigid anchoring elements for the end plates of adjacent vertebrae on its upper face and on its lower face, and a deformable structure in the area between upper face and lower face.

Accordingly, an implant in the form of a plug is used, which is inserted into the opening in the annulus and essentially has a deformable structure, so that it adapts to the structure of the annulus and installs itself, in particular, in the sealing area at the edge of the opening to be closed, tightly against the annulus tissue, while, on the other hand, not impairing the free movability of the annulus tissue. On the other hand, owing to the rigid anchoring elements on the upper face and on the lower face of the plug, this deformable plug can be secured to the end plates of the adjacent vertebrae, whereby the vertebrae ensure such positioning of the plug that it is not removed from its position by the movement of the annulus.

The rigid anchoring elements can be formed, for example, in accordance with a preferred embodiment, by the upper face and/or the lower face being more rigid, i.e., more stable with respect to their shape, than the area of the plug lying between upper face and lower face. In this case, the upper face and the lower face act as anchoring elements and form contact surfaces which rest against the end plates and thereby fix the plug in its position.

It is expedient for the implant to consist of resorbable material, so that the plug disintegrates after some time. Once the implanted cell cultures have grown in, they close the annulus again, so that the plug can then be replaced by the patient's own body tissue.

The plug may, in particular, consist of a porous material. The pore size may, for example, be between 150 μm and 200 μm. It is thus possible to also inject cell cultures into the plug so as to enable ingrowth of these in this area and accelerated closure of the opening.

It is particularly advantageous for the upper face and the lower face to carry at least one projection which is insertable into the end plate of the adjacent vertebra. These projections may engage in the material of the adjacent vertebrae and thereby fix the plug in the desired position. For example, it is possible to make corresponding recesses in the vertebra, in which these projections engage.

It is expedient for several projections to lie alongside one another in a line. These projections may, for example, then engage in a line-shaped recess in the end plate.

In another embodiment, provision is made for the projection to be a longitudinal rib.

Here it is advantageous for the line of projections or the longitudinal direction of the longitudinal rib to extend parallel to the direction of insertion of the plug. In this way, the plug can be pushed into the opening, and, at the same time, the projections arranged one behind the other in a line or the correspondingly aligned longitudinal rib engage in a channel extending in the direction of advance in the vertebra, which was made prior to the insertion into the vertebra. A very secure fixing of the plug is thereby obtained.

An additional fixing of the plug may be achieved by the projection having a toothing.

In particular, in the case of larger plugs it is expedient for several projections to be arranged alongside one another transversely to the direction of insertion of the plug, so as to enable a particularly homogenous fixing of the plug in the adjacent vertebrae.

Here it is advantageous for the projections to be set back from the side face of the plug, so that the portions of the plug projecting at the sides may adapt optimally to the tissue of the annulus as this tissue is flexible and possibly elastic.

In a preferred embodiment provision is made for the plug to have the shape of a cube or a rectangular parallelepiped. Other shapes are, however, also possible for the plug, for example, plugs in the shape of a cylinder whose longitudinal axis extends parallel to the direction of insertion may be used.

In a particularly preferred embodiment, the plug is covered with a membrane which also consists of resorbable material. This additionally seals the plug off from the outside, so that cells injected into it safely remain in the plug.

Here it is expedient for the membrane to be resorbable more slowly than the plug.

Furthermore, provision may be made for the plug to be provided with an anti-adhesion agent on its outer side so as to avoid formation of scar tissue in this area.

It is advantageous for the implant to be part of a set of plugs having a different height and/or width, so that during the surgical operation the operating surgeon may select a suitably dimensioned plug from this set and thereby achieve optimum sealing of the opening in the annulus fibrosus.

The following description of preferred embodiments of the invention serves in conjunction with the drawings to explain the invention in greater detail.

### BRIEF DESCRIPTION OF THE DRAWINGS

FIG. 1a shows a schematic plan view of the annulus fibrosus arranged between two vertebrae with an inserted implant for closure of an opening in the annulus fibrosus;

FIG. 1b shows a side view of the vertebrae of FIG. 1;

FIG. 2 shows a perspective view of the implant of FIGS. 1a and 1b with altered dimensions;

FIG. 3 shows a modified embodiment of an implant with several ribs for anchorage in the adjacent vertebrae;

FIG. 4 shows a schematic view of a vertebra and an annulus fibrosus with a prolapse of the intervertebral disc;

FIG. 5 shows a representation similar to FIG. 4 when opening the annulus fibrosus and removing excessive nucleus tissue;

FIG. 6 shows a view similar to FIG. 1 when inserting a test implant in the annulus opening;

FIG. 7 shows a view similar to FIG. 6 when inserting a chisel for preparing the end plates of the adjacent vertebra;

FIG. 8 shows a view similar to FIG. 1 when inserting an implant for closure of the annulus;

FIG. 9 shows a view similar to FIG. 4 with inserted implant; and

FIG. 10 shows a view similar to FIG. 9 when injecting cell material into the implant and into the nucleus.

### DETAILED DESCRIPTION OF THE INVENTION

As will be apparent from the drawings, in particular, from FIGS. 1a, 1b and 4, an intervertebral disc 3 is arranged between two vertebrae 1, 2. The intervertebral disc 3 consists of the central nucleus 4 and the annulus fibrosus 5 surrounding it. The upper vertebra 1 is omitted in FIG. 4 for the sake of clarity. It shall, however, be understood that this upper vertebra 1 rests with its lower end plate 6 in a surface-to-surface manner in the same way as the lower vertebra 2 with its upper end plate 7 against the intervertebral disc 3. FIG. 4 shows schematically how through an opening 8 in the annulus fibrosus, for example, in the form of a tear, nucleus material 9 leaks out from the nucleus 4. This leaking material can bear on adjacent nerve roots and cause pain.

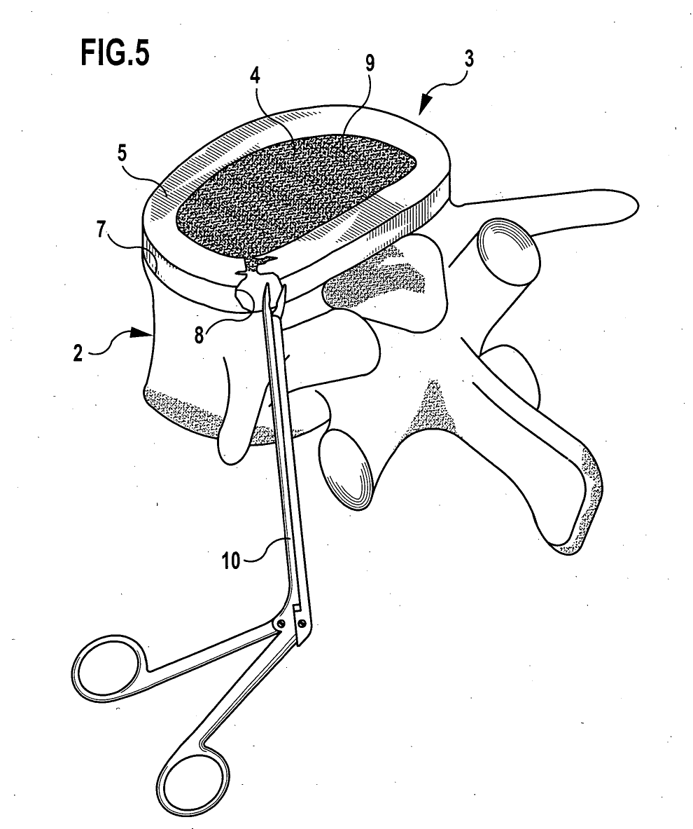

To repair this injury, the nucleus material 9 leaking from the opening 8 is removed in a surgical operation. This may be carried out with, for example, a forceps-like instrument 10 with which the jelly-like nucleus material 9 is cut off and the opening 8 may also be enlarged and its edges smoothed (FIG. 5). The thus removed nucleus material 9 may be used for growing a culture of the patient's own body cells. In this way, it is possible to produce, for example, within two weeks a cell culture which may then be used in the manner described hereinbelow.

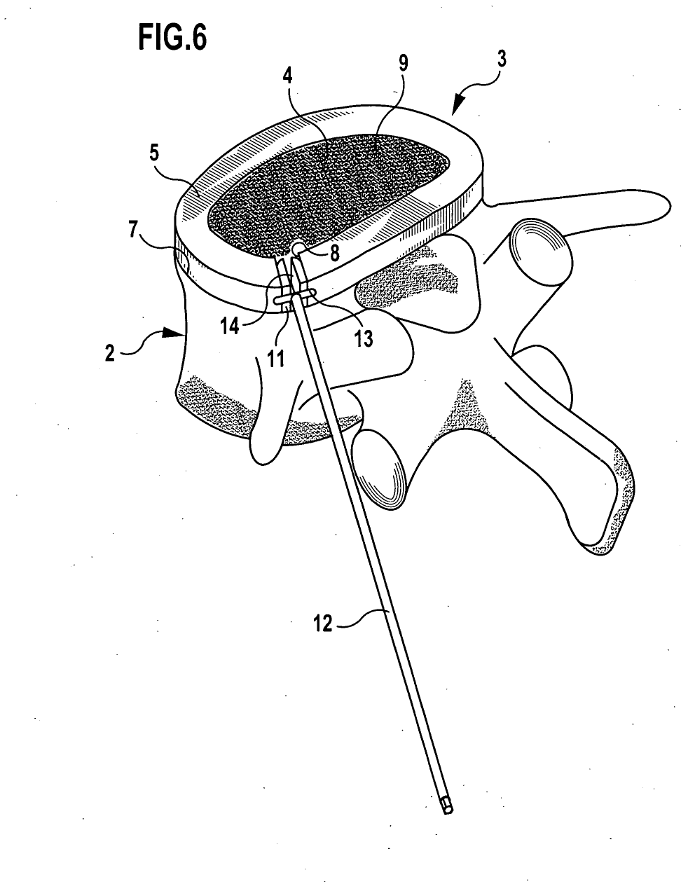

The opening 8 must be closed in a further surgical step. For this purpose, a test implant 11 is first inserted into the opening 8. The test implant 11 consists, for example, of a biocompatible metal and is selected in relation to the size of the opening 8 such that upon introducing the test implant 11 into the opening 8, the latter is not unnecessarily enlarged. The test implant 11 may have, for example, the shape of a relatively narrow rectangular parallelepiped. The test implant 11 is connectable (FIG. 6) by a rod 12 to a handle, not shown in the drawings, and has projections 13 at the sides, which delimit the penetration depth into the opening 8.

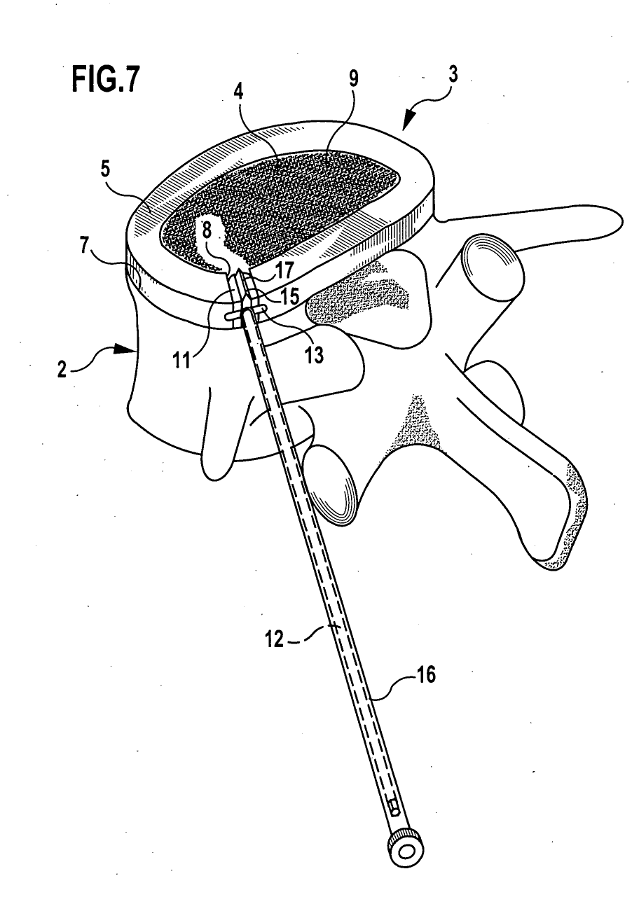

On its upper face and on its lower face, the test implant 11 has a central groove 14 extending parallel to the rod 12. The groove 14 forms a guide groove for a chisel 15. This chisel 15 is held at the end of a tubular shaft 16, which after removal of the handle from the rod 12, can be pushed over the rod 12 (FIG. 7). The chisel 15 is guided in the groove 14 and protrudes from it with a cutting edge 17. When the chisel 15 is advanced along the rod 12, it cuts with its cutting edge 17 a groove 18 in the adjacent end plate 6 of the vertebra 1. This groove then extends parallel to the groove 14 in the test implant 11.

This procedure may be carried out in the same way at the lower face of the test implant 11. In this way, a corresponding groove 19 is made there in the end plate 7 of the vertebra 2.

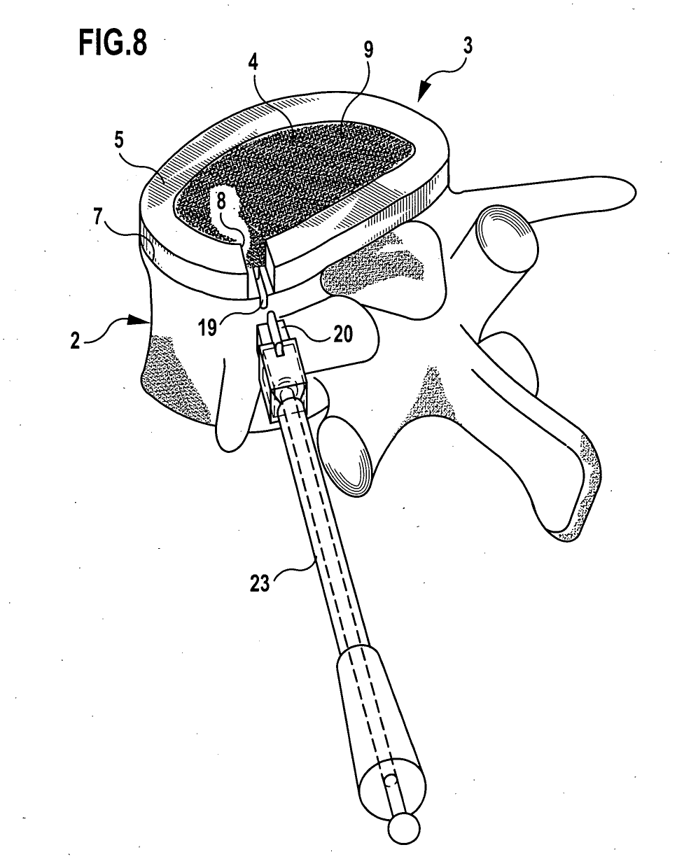

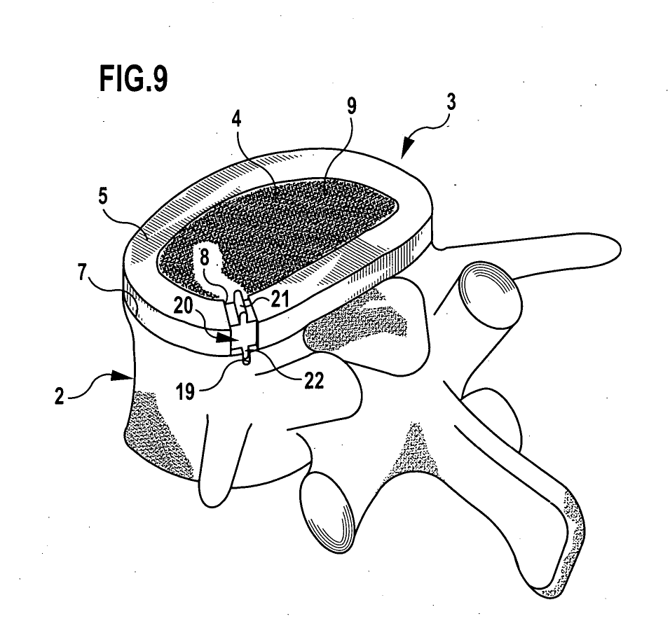

After removal of the test implant 11, an implant 20 may be inserted in the opening 8 in order to close it. In the embodiment shown in FIG. 8, the implant 20 has the shape of a rectangular parallelepiped which carries webs or ribs 21, 22 extending at the center of its upper face and its lower face and running parallel to the edges of the implant 20. The implant 20 is pushed into the opening 8 using a suitable insertion instrument 23 which releasably grips the implant 20. The direction of advance extends parallel to the longitudinal direction of the ribs 21, 22 which enter the grooves 18 and 19, respectively, during the insertion. Once the implant 20 has been pushed fully into the opening 8, the insertion instrument may be released and removed. As will be apparent from the illustration in FIG. 9, the implant 20 now closes the opening 8 and is supported at its upper face on the end plate 6 of the vertebra 1 and at its lower face on the end plate 7 of the vertebra 2. The ribs 21 and 22 engage the grooves 18 and 19, respectively, and thereby fix the implant 20 in this position.

The implant 20 consists of a resorbable material and is porous. The size of the pores lies between 150 μm and 200 μm. The material of the plug-shaped implant 20 is flexible and elastic, so that it adapts optimally to the contour of the opening 8 and also easily follows the movements of the material of the annulus fibrosus. The ribs 21 and 22, on the other hand, are rigid, i.e., considerably harder and not deformable or elastic in the same way as the rest of the material of the implant 20. This results in a rigid connection in the area in which the implant 20 is fixed to the adjacent vertebrae 1, 2, but in between the material of the implant 20 adapts to the movability of the material of the annulus fibrosus and reliably closes the opening 8.

In this way, the entire material of the plug between the ribs 21, 22 may be flexible or elastic, but it is also possible for the material of the plug to become progressively firmer towards the ribs 21, 22. For example, the upper face and the lower face of the plug could be rigid in a way similar to the ribs 21, 22, so that they form rigid contact surfaces at the end plates of the vertebrae. It is merely important that the material of the plug should be flexible and elastic in the central area thereof so as to ensure adaptation to the movability of the annulus fibrosus tissue.

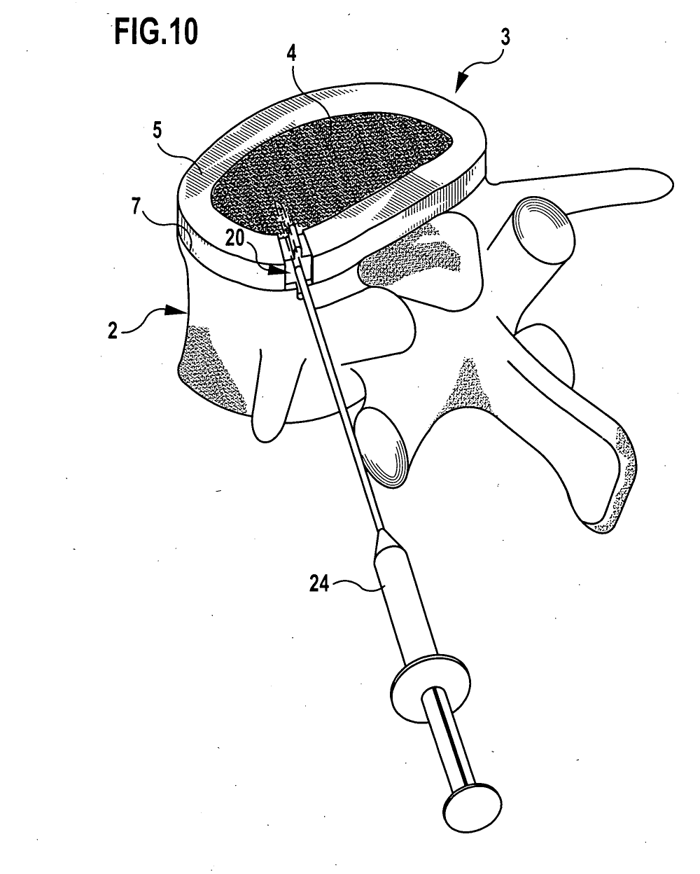

When the opening 8 in the annulus fibrosus 5 has been closed in this way, the cell material grown outside of the body can be introduced into the interior of the intervertebral disc 3. As a rule, this is carried out approximately two weeks after the surgical operation described above. The introduction may be carried out using an injection needle 24 (FIG. 10) which is inserted through the implant 20 into the interior of the intervertebral disc 3 and injects the material into both this interior and the porous area of the implant 20. The injected body cells grow in the interior of the intervertebral disc 3 and in the implant 20 and finally close the opening 8 reliably, so that the opening also remains closed when the implant 20 gradually disintegrates and is resorbed by the body.

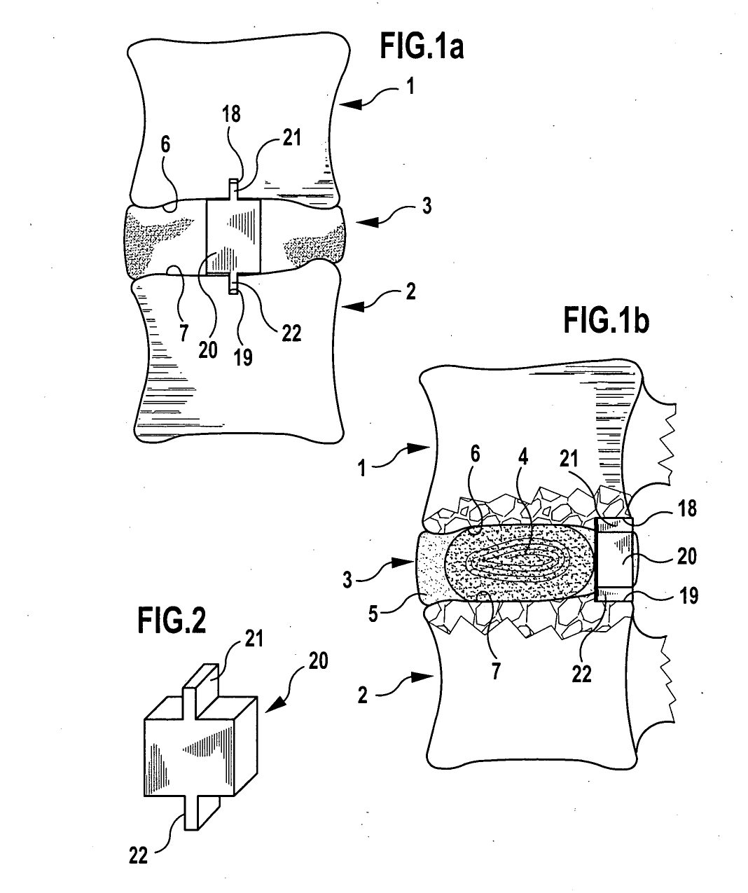

In the embodiment shown in FIGS. 1 to 10, the implant 20 has substantially the shape of a cube or a rectangular parallelepiped and carries on its upper face and on its lower face a web arranged at the center of each of these.

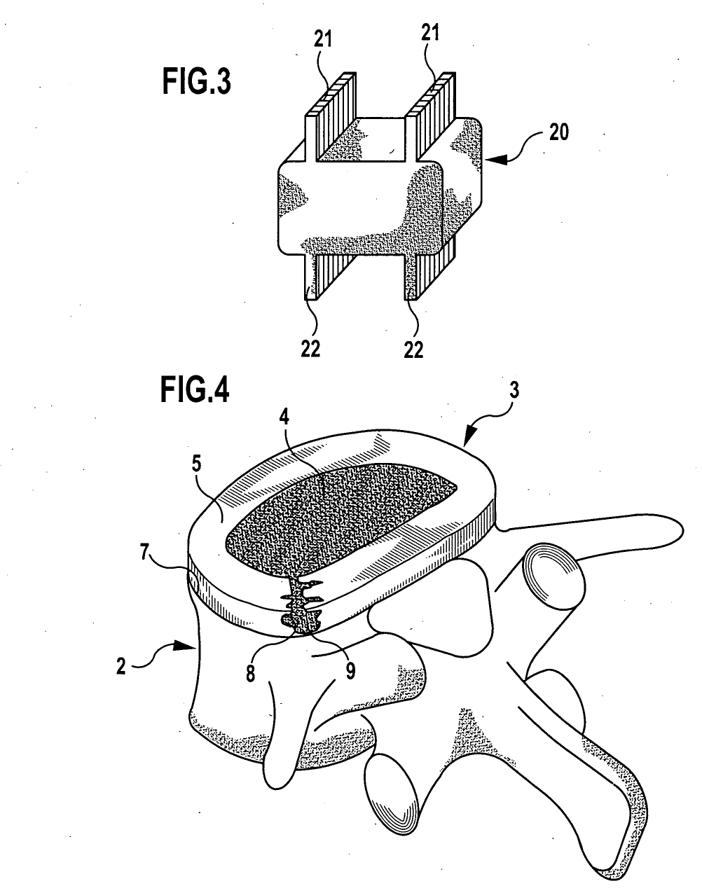

In the modified embodiment of FIG. 3, the implant 20 also has substantially the shape of a rectangular parallelepiped but is broader, and, therefore, two parallel ribs 21 and 22, respectively, are arranged on the upper face and the lower face, respectively. The ribs 21 and 22 are spaced from the side surfaces of the implant 20, so that in the area of the side surfaces the implant can adapt optimally to the tissue of the annulus fibrosus owing to the flexible and elastic structure, while the implant 20 is fixed by the parallel ribs 21, 22 in corresponding double grooves 18, 19 in the end plates of the vertebrae.

Instead of the ribs 21, 22, several pin-shaped projections lying alongside one another in a line could be provided. Furthermore, with a view to better fixing of the implant in the vertebrae, it is also possible to provide the ribs or projections with a toothing which engages in the side walls of the grooves 18, 19.

On its outer side, the implant 20 may be covered with a membrane which is impervious and seals the porous implant 20 off from the outside. This membrane may also consist of resorbable material. This material preferably decomposes more slowly than the material of the rest of the implant 20 and is, therefore, resorbed later.

Furthermore, an anti-adhesion layer, not shown in detail in the drawings, may be applied to this area. This may consist, for example, of a gelatin-containing gel or spray. Post-operative formation of scar tissue may thereby be prevented.

1. Implant in the form of a plug for closing an opening in the annulus fibrosus, said plug having rigid anchoring elements for the end plates of adjacent vertebrae on its upper face and on its lower face, and a deformable structure in the area between upper face and lower face.

2. Implant in accordance with claim 1, wherein the upper face and/or the lower face are more rigid than the areas of the plug lying between upper face and lower face thereof.

3. Implant in accordance with claim 1, said implant consisting of resorbable material.

4. Implant in accordance with claim 1, said plug consisting of porous material.

5. Implant in accordance with claim 4, the pore size of the porous material lying between 150 μm and 200 μm.

6. Implant in accordance with claim 1, wherein the upper face and the lower face carry at least one projection insertable in the end plate of the adjacent vertebra.

7. Implant in accordance with claim 6, wherein several projections lie alongside one another in a line.

8. Implant in accordance with claim 6, wherein the projection is a longitudinal rib.

9. Implant in accordance with claim 7, wherein the line of the projections or the longitudinal direction of the longitudinal ribs extends parallel to the direction of insertion of the plug.

10. Implant in accordance with claim 8, wherein the line of the projections or the longitudinal direction of the longitudinal ribs extends parallel to the direction of insertion of the plug.

11. Implant in accordance with claim 6, wherein the projection has a toothing.

12. Implant in accordance with claim 6, wherein several projections are arranged alongside one another transversely to the direction of insertion of the plug.

13. Implant in accordance with claim 11, wherein the projections are set back from the side surface of the plug.

14. Implant in accordance with claim 1, said plug having the shape of a cube or a rectangular parallelepiped.

15. Implant in accordance with claim 3, wherein the plug is covered with a membrane which also consists of resorbable material.

16. Implant in accordance with claim 14, wherein the membrane is resorbable more slowly than the plug.

17. Implant in accordance with claim 1, said plug being provided with an anti-adhesion agent on the outer side thereof.

18. Implant in accordance with claim 1, said implant being part of a set of plugs having a different height and/or width.
